(19) United States
(12) Patent Application Publication
(10) Pub. No.: US 2004/0115690 A1
(43) Pub. Date: Jun. 17, 2004

(54) Method of detecting inflammatory lung disorders

(76) Inventors: Luca Rastelli, Guilford, CT (US); Glennda Smithson, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/613,105

(22) Filed: Jul. 2, 2003

Related U.S. Application Data
(62) Division of application No. 09/865,812, filed on May 25, 2001, now abandoned.
(60) Provisional application No. 60/207,104, filed on May 25, 2000.

(51) Int. Cl. C12Q 1/68

(52) U.S. Cl. 435/6

(57) ABSTRACT

Disclosed are methods of detecting and treating inflammatory lung disorders, such as emphysema, asthma bronchitis or allergy. Also disclosed are methods of identifying agents for treating inflammatory lung disorders.

### RELATED APPLICATIONS

This application is a divisional of co-pending application Ser. No. 09/865,812, filed May 25, 2001, which claims priority to U.S. S No. 60/207,104, filed May 5, 2000, each of which is incorporated herein by reference in its entirety.

### FIELD OF THE INVENTION

The invention relates to methods of detecting inflammatory lung disorders.

### BACKGROUND OF THE INVENTION

Antileukoproteases, also known as secretory leukocyte protease inhibitors, are a class of acid-stable proteinase inhibitors with strong affinity for trypsin and chymotrypsin as well as for neutrophil lysosomal elastase and cathepsin G. Antileukoproteases are present in mucous fluids such as seminal plasma, cervical mucus, bronchial and nasal secretions, and tears.

### SUMMARY OF THE INVENTION

In various aspects the invention includes methods of diagnosing an inflammatory lung disorder such as emphysema, asthma, bronchitis and allergy by measuring the expression of a nucleic acid encoding an antileukoprotease polypeptide in a test cell population and comparing the expression of the nucleic acid to the expression of a nucleic acid encoding an antileukoprotease polypeptide in reference profile. Examples of antileukoprotease nucleic acids and polypeptides are illustrated in SEQ ID NO:1-2. The reference profile can be a inflammation positive reference profile or an inflammation negative reference profile. An inflammation positive profile is a profile including cells primarily with an inflammatory lung disorder. In contrast an inflammation negative reference profile is a profile including cells primarily without an inflammatory lung disorder. A similarity between the expression of the nucleic acid in the test cell population and the inflammation positive reference profile indicates the presence of a lung inflammatory disorder in the mammal. An increase in expression of the nucleic acid in the test cell population and the inflammation negative reference profile indicates the presence of a lung inflammatory disorder in the mammal.

In a further aspect, the invention provides methods treating or preventing an inhibits antileukoprotease. Compounds that inhibit antileukoprotease include a compound that binds antileukoprotease nucleic acids or polypeptides. Examples of compounds that bind antleukoprotease nucleic acids or polypeptides include antileukoprotease antisense nucleic acid, ribozymes, and antibodies.

Also provided are a methods of identifying a compound that inhibits lung inflammation, by providing a cell expressing antileukoprotease, contacting the cell with a test compound and measuring the expression of antileukoprotease. A decrease in expression in the presence of the test compound compared to that in the absence of the test compound indicates that test compound inhibits lung inflammation. Also inlcuded in the invention are compounds identified by the method.

In yet a futher aspect, the invention provides a method of assessing the prognosis of a subject with a cancer, such as thyroid carcinoma, ovarian carcinoma or renal cell carcinoma. by measuring the expression of a nucleic acid encoding an antileukoprotease polypeptide in a test cell population and comparing the expression of the nucleic acid to the expression of a nucleic acid encoding an antileukoprotease polypeptide in a cancer reference profile. A cancer reference profile includes primarily cancerous cells. A substantial similarity between the expression of the nucleic acid sequence in test cell population and the cancer reference profile indicates an adverse prognosis of the subject.

In still a further aspect, the invention provides a method of assessing the metastatic potential of tumor, such as a thyroid tumor, by measuring the expression of a nucleic acid encoding an antileukoprotease polypeptide in a subject derived cell population and comparing the expression of the nucleic acid to the expression of a nucleic acid encoding an antileukoprotease polypeptide in a metastatic cancer reference profile. A metastatic cancer reference profile includes cells in which the metatstatic potentional is known. a substantial similarity between the expression of the nucleic acid sequence in the subject derived cell population and the metastatic reference profile indicates the tumor is metastatic.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1. is a histogram illustrating the overexpression of antileukoprotease in ovarian carcinoma cells lines compared to normal ovary.

FIG. 2. is a histrogram illustrating the overexpression of antileukoprotease in thyroid tumors compared to normal thyoid or normal adjacent tissue.

FIG. 3. is table illustrating the SAGE library data results illustrating overexpression of antileukoprotease in ovarian tumors compared to normal ovary.

### DETAILED DESCRIPTION

The present invention is based in part on the discovery of changes in expression pattern of antileukoprotease nucleic acid is up-regulated in certain cancer cells and lung cells.

The change is expression pattern was identified by GeneCalling™ analysis (U.S. Pat. No. 5,871,697; Shimkets et al., 1999 Nature Biotechnology 17:198-803, incorporated herein by reference in their entireties), TaqMan™ and SAGE analysis. Analysis of numerous normal and tumor samples revealed that antileukoprotease is up-regulated in metastatic vs non-metastatic thyroid cancer, overexpressed in ovarian carcinomas tumors and tumor derived cell lines compared with normal ovary and overexpressed in kidney and thyroid carcinoma tissues compared with normal adjacent tissues (NATs) obtained during surgery and normal tissues.

In its various aspects and embodiments, the invention includes providing a test cell population which includes at least one cell that is capable of expressing antileuktoprotease. By “capable of expressing” is meant that the gene is present in an intact form in the cell and can be expressed. Expression of the antileukoprotease sequences is then detected, if present, and, preferably, measured. Using sequence information provided by the database entries for known antileukoprotease sequences or the sequence information disclosed herein, e.g., SEQ ID NO: 1 or SEQ NO:2 expression of the antileukoprotease sequences can be detected (if present) and measured using techniques well known to one of ordinary skill in the art. For example, sequences within the sequence database entries corresponding to antileukoprotease sequences, or within the sequences disclosed herein, can be used to construct probes for detecting antileukoprotease RNA sequences in, e.g., northern blot hybridization analyses or methods which specifically, and, preferably, quantitatively amplify specific nucleic acid sequences. As another example, the sequences can be used to construct primers for specifically amplifying the antileukoprotease sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

Expression can be also measured at the protein level, i.e., by measuring the levels of polypeptides encoded by the gene products described herein. Such methods are well known in the art and include, e.g., immunoassays based on antibodies to proteins encoded by the genes.

Expression level of the antileukoprotease sequences in the test cell population is then compared to expression levels of the sequences in one or more cells from a reference profile. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17:798-803.

A reference profile is an expression pattern derived from a single reference population or from a plurality of expression patterns. The reference profile can be a database of expression patterns from previously tested cells for which one of the herein-described conditions (e.g., inflammatory lung disorder, metastatic state or cancer) is known.

In some embodiments, the test cell will be included in a cell sample from a subject known to contain, or to be suspected of containing, inflammatory lung cells or tumorous cells. In other embodiments, the cell sample will be derived from a subject from a region known to contain, or suspected of containing, a metastasis of a primary tumor, such as a thyroid carcinoma.

The test cell is obtained from a bodily fluid, e.g., biological fluid (such as blood, serum, urine, saliva, milk, ductal fluid, or tears). For example, the test cell is purified from blood or another tissue.

Preferably, cells in the reference profile are derived from a tissue type as similar as possible to test cell, e.g., lung tissue. In some embodiments, the control cell is derived from the same subject as the test cell, e.g., from a region proximal to the region of origin of the test cell. In some embodiments, the test cell population is compared to multiple reference profiles. Each of the multiple reference profiles may differ in the known parameter or condition. Thus, a test cell population may be compared to a first reference profile known to have an inflammatory lung disorder, as well as a second reference population known not to have an inflammatory disorder.

Whether or not comparison of the gene expression profile in the test cell population to the reference profile reveals the presence, or degree, of the measured condition depends on the composition of the reference profile. For example, if the profile is composed of cells that have an inflammatory lung disorder, a similar gene expression level in the test cell population and a reference profile indicates the presence of the inflammatory disorder in the test cell population. Conversely, if the reference profile is composed of cells that do not have an inflammatory lung disorder, a similar gene expression profile between the test cell population and the reference profile indicates the absence of the inflammatory disorder in the test cell population

In various embodiments, the antileukoprotease sequence in a test cell population is considered comparable in expression level to the expression level of the antileukoprotease sequence if its expression level varies within a factor of 2.0, 1.5, or 1.0 fold to the level of the antileukoprotease transcript in the reference profile. In various embodiments, a antileukoprotease sequence in a test cell population can be considered altered in levels of expression if its expression level varies from the reference cell population by more than 1.0, 1.5, 2.0 or more fold from the expression level of the corresponding antileukoprotease sequence in the reference cell population.

If desired, comparison of differentially expressed sequences between a test cell population and a reference profile can be done with respect to a control nucleic acid whose expression is independent of the parameter or condition being measured. Expression levels of the control nucleic acid in the test and reference nucleic acid can be used to normalize signal levels in the compared populations.

The subject is preferably a mammal. The mammal can be, e.g., a human, non-human primate, mouse, rat, dog, cat, horse, or cow.

Diagnosing an Inflammatory Lung Disorder

The invention provides a method of diagnosing a inflammatory lung disorder, e.g., emphysema, asthma, bronchitis or inflammation of the small airway epithelium a subject. A inflammatory lung disorder is diagnosed by examining the expression of a nucleic acid encoding antileukoprotease from a test population of cells from a subject suspect of having an inflammatory lung disorder. The population of cells may contain cell of the lung, or may alternatively contain cells the respiratory system, such as cells of the airway epithelium.

Expression of a nucleic acid encoding antileukoprotease is measured in the test cell and compared to the expression of the sequence in the reference profile. A reference profile can be a inflammation positive reference profile. By “inflammation positive reference profile” is meant that the reference profile contains cells derived from tissues with a inflammatory lung disorder. Alternatively, the reference profile can be an inflammation negative reference profile. By “inflammation negative reference profile” is meant that the reference profile contains cells derived from tissues without an inflammatory lung disorder.

When a reference profile is an inflammation positive reference profile, a similarity in expression between antileukoprotease sequences in the test population and the reference profile indicates the presence of an inflammatory disorder in the subject. Conversely, a decrease in expression in the test cell population between antileukoprotease sequences in the test population and the inflammmation positive reference profile indicates the absence of an inflammatory disorder in the subject.

When the reference profile is an inflammation negative reference profile, a increase in expression pattern between the test cell population and the inflammation negative reference profile indicates the presence of inflammatory lung disorder. Conversely, a similarity in expression expression between antileukoprotease sequences in the test population and the inflammation negative reference profile indicates the absence of an inflammatory disorder in the subject.

Methods of Treating Disorders Associated with Aberrant Antileukoprotease Expression

The invention provides a method for treating disorders associated with aberrant antileukoprotease expression in a subject. Administration can be prophylactic or therapeutic to a subject at risk of (or susceptible to) an inflammatory lung disorder. The inflammatory lung disorder can be, e.g., emphysema, asthma, bronchitis or inflammation of the small airway epithelium. Alternatively, administration can be to a subject at risk of (or susceptible to) a disorder associated with aberrant expression or activity antileukoprotease, e.g., cancer such as thyroid carcinoma, ovarian carcinoma or renal cell carcinoma.

The therapeutic method includes decreasing or inhibiting the expression, or function, or antileukoprotease in the diseased cell relative to normal cells of the tissue type from which the diseased cells are derived. In these methods, the subject is treated with an effective amount of a compound, which decreases the amount of antileukoprotease in the subject. Administration can be systemic or local, e.g., in the immediate vicinity of, the subject's diseased cells. Expression can be inhibited in any of several ways known in the art. For example, expression can be inhibited by administering to the subject a nucleic acid that inhibits, or antagonizes, the expression of the antileukoprotease. In one embodiment, an antisense oligonucleotide can be administered which disrupts expression of antileukoprotease.

Alternatively, function antileukoprotease can be inhibited by administering a compound that binds to or otherwise inhibits the function of the gene products. The compound can be, e.g., an antibody to antileukoprotease.

These modulatory methods can be performed ex vivo or in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity antileukoprotease proteins or nucleic acid molecules. In one embodiment, the method involves administering an agent (e.g., an agent identified by a screening assay described herein), or combination of agents that modulates (e.g., upregulates or downregulates) expression or activity of antileukoprotease. In another embodiment, the method involves administering a protein or combination of proteins or a nucleic acid molecule or combination of nucleic acid, molecules as therapy to compensate for aberrant expression or activity of antileukoprotease nucleic acid.

Therapeutics that may be utilized include, e.g., (i) a polypeptide, or analogs, derivatives, fragments or homologs thereof of the overexpressed sequence; (ii) antibodies to the overexpressed sequence; (iii) antisense nucleic acids or nucleic acids that are “dysfunctional” (i.e., due to a heterologous insertion within the coding sequences of coding sequences of one or more overexpressed or underexpressed sequences); or (v) modulators (i.e., inhibitors, agonists and antagonists that alter the interaction between an overexpressed polypeptide and its binding partner. The dysfunctional antisense molecules are utilized to “knockout” endogenous function of a polypeptide by homologous recombination (see, e.g., Capecchi, Science 244: 1288-1292 1989)

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Administration of a prophylactic agent can occur prior to the manifestation of symptoms characteristic of aberrant gene expression, such that a disease or disorder is prevented or, alternatively, delayed in its progression. Depending on the type of aberrant expression detected, the agent can be used for treating the subject. The appropriate agent can be determined based on screening assays described herein.

Screening Assays for Identifying a Compound that Inhibit Lung Inflammation

In one aspect, the invention provides a method of identifying a compound that inhibit lung inflammation. The compound can be identified by providing a cell population that includes cells capable of expressing antileukoprotease. Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent.

An decrease in expression of the nucleic acid sequence in the test cell population compared to the expression of the nucleic acid sequence in the reference cell population that has not been exposed to the test agent indicates the test agent inhibits inflammation.

The test agent can be a compound not previously described or can be a previously known compound but which is not known to be an anti-inflammatory agent.

The invention also includes a compound identified according to this screening method.

Assessing the Prognosis of a Subject with a Cancer

Also provided is a method of assessing the prognosis of a subject with cancer, e.g., thyroid carcinoma, ovarian carcinoma or renal cell carcinoma by comparing the expression antileukoprotease in a test cell population to the expression of the sequences in a reference profile derived from patients over a spectrum of disease stages. By comparing gene expression of antileukoprotease in the test cell population and the reference profile, or by comparing the pattern of gene expression overtime in test cell populations derived from the subject, the prognosis of the subject can be assessed.

The reference profile includes primarily noncancerous or cancerous cells. A reference profile which includes primarily noncancerous cells is a non-cancer reference profile. A reference profile which includes primarily cancerous cells is a cancer reference profile. In some embodiments the cancer reference profile includes primarily disseminated cancerous cells. When the reference profile includes primarily noncancerous cells, an increase of expression of antileukoprotease in the test cell population, indicates less favorable prognosis. Conversely, when the reference profile includes primarily cancerous cells, an decrease of expression of antileukoprotease in the test cell population, indicates more favorable prognosis.

Assessing Metastatic Potential of a Tumor

In another aspect, the invention provides a method of assessing the mestastatic potential of a tumor, e.g., thyroid tumor, ovarian tumor or a renal cell tumor, in a subject by comparing levels of antileukoprotease sequence in a test and reference profile.

To assess metastatic potential, a test cell population is taken from the subject previously diagnosed with a tumor and antileuoprotease expression is measured. By comparing gene expression of antileukoprotease in the test cell population and the reference profile, the metastatic potential can be assessed.

The reference profile includes primarily cancerous cells of known metastatic potential. Accordingly, a similarity of expression of antileukoprotease in a test cell relative to a reference profile which includes primarily metatstatic cancerous cells indicates the tumor is metastatic. Conversely, when the reference profile includes primarily non-metastatic cancerous cells a similarity of expression of antileukoprotease in a test cell relative to the reference profile indicates the tumor is not metastatic.

If desired, expression of antileukoprotease can be measured along with expression level of other sequences whose expression is known to be altered according to metastatic potential.

Pharmaceutical Compositions

In another aspect the invention includes pharmaceutical, or therapeutic, compositions containing one or more therapeutic compounds described herein. Pharmaceutical formulations may include those suitable for oral, rectal, nasal, topical (including buccal and sub-lingual), vaginal or parenteral (including intramuscular, sub-cutaneous and intravenous) administration, or for administration by inhalation or insufflation. The formulations may, where appropriate, be conveniently presented in discrete dosage units and may be prepared by any of the methods well known in the art of pharmacy. All such pharmacy methods include the steps of bringing into association the active compound with liquid carriers or finely divided solid carriers or both as needed and then, if necessary, shaping the product into the desired formulation.

Pharmaceutical formulations suitable for oral administration may conveniently be presented as discrete units, such as capsules, cachets or tablets, each containing a predetermined amount of the active ingredient; as a powder or granules; or as a solution, a suspension or as an emulsion. The active ingredient may also be presented as a bolus electuary or paste, and be in a pure form, i.e., without a carrier. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration may be presented as a suppository with the usual carriers such as cocoa butter or polyethylene glycol. Formulations for topical administration in the mouth, for example buccally or sublingually, include lozenges, comprising the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents. Liquid sprays are conveniently delivered from pressurized packs.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insuffiator.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the compositions may be administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The pharmaceutical composition preferably is administered orally or by injection (intravenous or subcutaneous), and the precise amount administered to a subject will be the responsibility of the attendant physician. However, the dose employed will depend upon a number of factors, including the age and sex of the subject, the precise disorder being treated, and its severity. Also the route of administration may vary depending upon the condition and its severity.

### EXAMPLES

### Example 1

### Expression Analysis of Antileukoprotease in Various Tissues

The quantitative expression of antileukoprotease (GenBank Accession No: X04470; Table 1; SEQ ID NO:1-2) was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), and Panel 4 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for 13-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tₘ=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDR1). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDR1 or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table 4). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDR1) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 μg/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDR1. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C. The results of panel 4 suggest antileukoprotease nucleic acids and polypeptides are useful in the diagnosis of inflammatory lung disorders. The results further suggest, that inhibitors of antileukoprotease are useful in the treatment of inflammatory lung disorders.

The TaqMan™ expression profiles of the antileukoprotease transcript were generated using the X04470-specific Ag 588 set of forward (F) and reverse (R) primers, and probe (P) as shown in Table 2

TABLE 1
Nucleic Acid and Polypeptide Sequence of Human mRNA for antileukoprotease

| | | |
|---|---|---|
| 1 | gtcactcctg ccttcaccat gaagtccagc ggcctcttcc ccttcctggt gctgcttgcc | (SEQ ID NO:1) |
| 61 | ctgggaactc tggcaccttg ggctgtggaa ggctctggaa agtccttcaa agctggagtc | |
| 121 | tgtcctccta agaaatctgc ccagtgcctt agatacaaga aacctgagtg ccagagtgac | |
| 181 | tggcagtgtc cagggaagaa gagatgttgt cctgacactt gtggcatcaa atgcctggat | |
| 241 | cctgttgaca ccccaaaccc aacaaggagg aagcctggga agtgcccagt gacttatggc | |
| 301 | caatgtttga tgcttaaccc ccccaatttc tgtgagatgg atggccagtg caagcgtgac | |
| 361 | ttgaagtgtt gcatgggcat gtgtgggaaa tcctgcgttt cccctgtgaa agcttgattc | |
| 421 | ctgccatatg gaggaggctc tggagtcctg ctctgtgtgg tccaggtcct ttccaccctg | |
| 481 | agacttggct ccaccactga tatcctcctt tggggaaagg cttggcacac agcaggcttt | |
| 541 | caagaagtgc cagttgatca atgaataaat aaacgagcct atttctcttt gcac | |

| | |
|---|---|
| MKSSGLFPFLVLLALGTLAPWAVEGSGKSFKAGVCPPKKSAQCLRYKKPECQSDWQCPGKKRCCPDTC | (SEQ ID NO:2) |
| GIKCLDPVDTPNPTRRKPGKCPVTYGQCLMLNPPNFCEMDGQCKRDLKCCMGMCGKSCVSPVKA | |

| | | |
|---|---|---|
| Ag 588 (F): | 5′-TGCCTTCACCATGAAGTCCA-3′ | (SEQ ID NO:3) |
| Ag 588 (R): | 5′-AGCCCAAGGTGCCAGAGTT-3′ | (SEQ ID NO:4) |
| Ag 588 (P): | FAM-5′-CTTCCTGGTGCTGCTTGCCCTGG-3′TAMRA | (SEQ ID NO:5) |

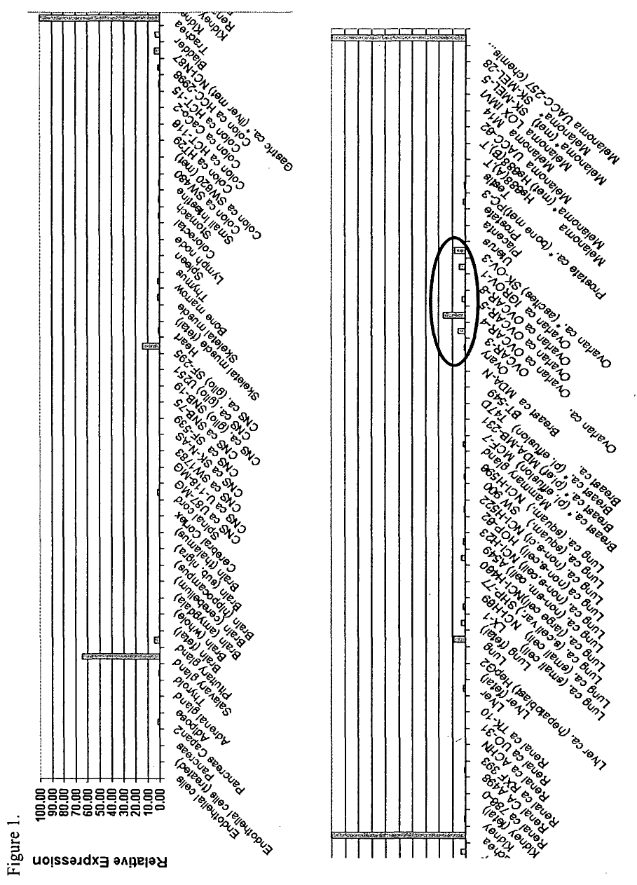

The results shown in FIG. 1 relate to 41 normal human tissues and 55 human cancer cell lines and demonstrate the overexpression of X04470 in ovarian carcinoma cell lines compared with normal ovary.

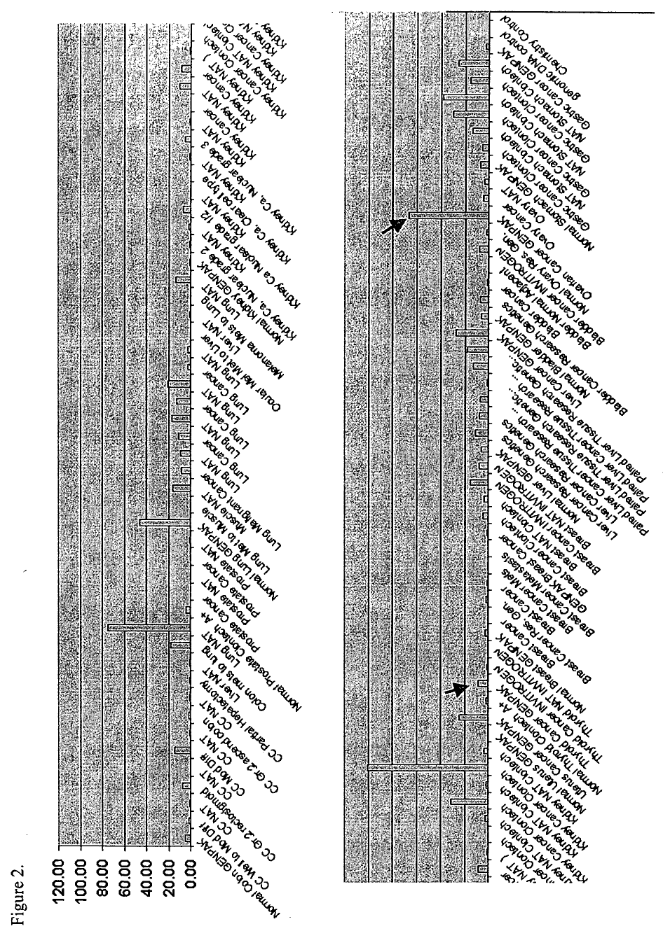

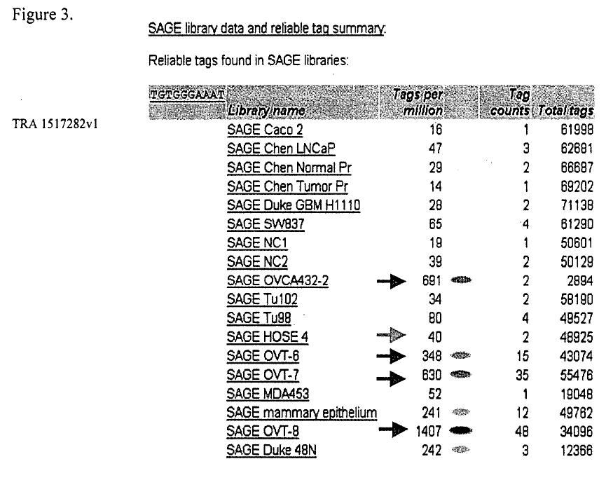

The results shown in FIG. 2 relate to additional tumor tissues, many of which are matched with normal adjacent tissue (NAT), as defined by the operating surgeon that obtained the samples. FIG. 3 illustrates that antileukoprotease X04470 is overexpressed in thyroid tumors compared either with normal thyroid or NAT. This analysis corroborates the GeneCalling™ results which originally identified the expression of X04470. Antileukoprotease is also overexpressed in some of the kidney carcinoma tissues compared with the corresponding NATs and 1 of 2 ovarian carcinomas compared with normal ovary suggesting that antileukoprotease plays an important role in tumorigenesis in various carcinomas, includingovarian carcinomas.

NCI's CGAP Serial Analysis of Gene Expression (SAGE) (FIG. 2) indicates that antileukoprotease is upregulated in ovarian tumors vs normal ovary.

TABLE 3
Taq Man results for PANEL 1

| | Rel. Expr., % |
|---|---|
| Tissue Name | tm688f_ag588 |
| Adipose | 0.3 |
| Adrenal gland | 0.1 |
| Bladder | 3.6 |
| Brain (amygdala) | 0 |
| Brain (cerebellum) | 0 |
| Brain (hippocampus) | 0 |
| Brain (substantia nigra) | 0.1 |
| Brain (thalamus) | 0 |
| Cerebral Cortex | 0 |
| Brain (fetal) | 0 |
| Brain (whole) | 0 |
| CNS ca. (glio/astro) U-118-MG | 0 |
| CNS ca. (astro) SF-539 | 0 |
| CNS ca. (astro) SNB-75 | 0.2 |
| CNS ca. (astro) SW1783 | 0 |
| CNS ca. (glio) U251 | 0 |
| CNS ca. (glio) SF-295 | 13.8 |
| CNS ca. (glio) SNB-19 | 0 |
| CNS ca. (glio/astro) U87-MG | 0 |
| CNS ca.* (neuro; met) SK-N-AS | 0 |
| Mammary gland | 1.8 |
| Breast ca. BT-549 | 0 |
| Breast ca. MDA-N | 0 |
| Breast ca.* (pl. effusion) T47D | 0 |
| Breast ca.* (pl. effusion) MCF-7 | 0 |
| Breast ca.* (pl. ef) MDA-MB-231 | 0 |
| Small intestine | 0.4 |
| Colorectal | 0.4 |
| Colon ca. HT29 | 0 |
| Colon ca. CaCo-2 | 0 |
| Colon ca. HCT-15 | 0.4 |
| Colon ca. HCT-116 | 0 |
| Colon ca. HCC-2998 | 1.4 |
| Colon ca. SW480 | 0 |
| Colon ca.* (SW480 met) SW620 | 0 |
| Stomach | 1.1 |
| Gastric ca.* (liver met) NCI-N87 | 4.3 |
| Heart | 0.9 |
| Fetal Skeletal | 0 |
| Skeletal muscle | 1.5 |
| Endothelial cells | 0 |
| Endothelial cells (treated) | 0 |
| Kidney | 0.6 |
| Kidney (fetal) | 0 |
| Renal ca. 786-0 | 0 |
| Renal ca. A498 | 1 |
| Renal ca. ACHN | 0 |
| Renal ca. TK-10 | 0 |
| Renal ca. UO-31 | 0 |
| Renal ca. RXF 393 | 0 |
| Liver | 1.6 |
| Liver (fetal) | 0.2 |
| Liver ca. (hepatoblast) HepG2 | 0 |
| Lung | 8.6 |
| Lung (fetal) | 3 |
| Lung ca. (non-s. cell) HOP-62 | 0.5 |
| Lung ca. (large cell) NCI-H460 | 2.6 |
| Lung ca. (non-s. cell) NCI-H23 | 0.2 |
| Lung ca. (non-s. cl) NCI-H522 | 0 |
| Lung ca. (non-sm. cell) A549 | 1.6 |
| Lung ca. (s. cell var.) SHP-77 | 0 |
| Lung ca. (small cell) LX-1 | 1.6 |
| Lung ca. (small cell) NCI-H69 | 0.1 |
| Lung ca. (squam.) SW 900 | 0.4 |
| Lung ca. (squam.) NCI-H596 | 0 |
| Lymph node | 0.3 |
| Spleen | 0 |
| Thymus | 0.2 |
| Ovary | 0.5 |
| Ovarian ca. IGROV-1 | 4.6 |
| Ovarian ca. OVCAR-3 | 5.4 |
| Ovarian ca. OVCAR-4 | 16.5 |
| Ovarian ca. OVCAR-5 | 2.6 |
| Ovarian ca. OVCAR-8 | 0.2 |
| Ovarian ca.* (ascites) SK-OV-3 | 7.9 |
| Pancreas | 0.4 |
| Pancreatic ca. CAPAN 2 | 1.2 |
| Pituitary gland | 4.3 |
| Plancenta | 0 |
| Prostate | 1.2 |
| Prostate ca.* (bone met) PC-3 | 0.6 |
| Salavary gland | 64.2 |
| Trachea | 100 |
| Spinal cord | 1.6 |
| Testis | 0.3 |
| Thyroid | 0.4 |
| Uterus | 0.4 |
| Melanoma M14 | 0 |
| Melanoma LOX IMVI | 0 |
| Melanoma UACC-62 | 0 |
| Melanoma SK-MEL-28 | 0 |
| Melanoma* (met) SK-MEL-5 | 0 |
| Melanoma Hs688(A).T | 0 |
| Melanoma* (met) Hs688(B).T | 0 |

TABLE 4
Taq Man Results for Panel 2

| | Rel. | Rel. |
|---|---|---|
| | Expr., % | Expr., % |
| | 2Dtm2303f_{—} | 2Dtm2339f_{—} |
| Tissue Name | ag588 | ag588 |
| Normal Colon GENPAK 061003 | 4.8 | 4.4 |
| 83219 CC Well to Mod Diff (ODO3866) | 1.3 | 1.2 |
| 83220 CC NAT (ODO3866) | 0.9 | 0.8 |
| 83221 CC Gr. 2 rectosigmoid (ODO3868) | 1.8 | 1.8 |
| 83222 CC NAT (ODO3868) | 0 | 0 |
| 83235 CC Mod Diff (ODO3920) | 3.1 | 2.9 |
| 83236 CC NAT (ODO3920) | 0.5 | 0.4 |
| 83237 CC Gr. 2 ascend colon (ODO3921) | 2.3 | 1.8 |
| 83238 CC NAT (ODO3921) | 0.4 | 0.4 |
| 83241 CC from Partial Hepatectomy | 1.8 | 2 |
| (ODO4309) | | |
| 83242 Liver NAT (ODO4309) | 2.4 | 2.2 |
| 87472 Colon mets to lung (OD04451-01) | 5.3 | 5.1 |
| 87473 Lung NAT (OD04451-02) | 32.8 | 35.8 |
| Normal Prostate Clontech A+ 6546-1 | 5 | 4.8 |
| 84140 Prostate Cancer (OD04410) | 0.3 | 0.2 |
| 84141 Prostate NAT (OD04410) | 0.2 | 0.2 |
| 87073 Prostate Cancer (OD04720-01) | 0.7 | 0.8 |
| 87074 Prostate NAT (OD04720-02) | 1.8 | 1.5 |
| Normal Lung GENPAK 061010 | 56.3 | 55.1 |
| 83239 Lung Met to Muscle (ODO4286) | 0 | 0 |
| 83240 Muscle NAT (ODO4286) | 24.5 | 26.1 |
| 84136 Lung Malignant Cancer (OD03126) | 42 | 41.8 |
| 84137 Lung NAT (OD03126) | 40.3 | 42.9 |
| 84871 Lung Cancer (OD04404) | 27.4 | 28.9 |
| 84872 Lung NAT (OD04404) | 42.6 | 39.2 |
| 84875 Lung Cancer (OD04565) | 13.7 | 12.8 |
| 84876 Lung NAT (OD04565) | 18.3 | 18.4 |
| 85950 Lung Cancer (OD04237-01) | 6.4 | 6.2 |
| 85970 Lung NAT (OD04237-02) | 12.8 | 12.2 |
| 83255 Ocular Mel Met to Liver | 0 | 0 |
| (ODO4310) | | |
| 83256 Liver NAT (ODO4310) | 3.6 | 3.6 |
| 84139 Melanoma Mets to Lung | 0.4 | 0.4 |
| (OD04321) | | |
| 84138 Lung NAT (OD04321) | 77.9 | 76.3 |
| Normal Kidney GENPAK 061008 | 1.6 | 1.5 |
| 83786 Kidney Ca, Nuclear grade 2 | 3.3 | 3.2 |
| (OD04338) | | |
| 83787 Kidney NAT (OD04338) | 3 | 3 |
| 83788 Kidney Ca Nuclear grade 1/2 | 6.7 | 6.7 |
| (OD04339) | | |
| 83789 Kidney NAT (OD04339) | 0.7 | 0.6 |
| 83790 Kidney Ca, Clear cell type | 0 | 0 |
| (OD04340) | | |
| 83791 Kidney NAT (OD04340) | 2.5 | 2.3 |
| 83792 Kidney Ca, Nuclear grade 3 | 7.1 | 6.8 |
| (OD04348) | | |
| 83793 Kidney NAT (OD04348) | 1.8 | 1.8 |
| 87474 Kidney Cancer (OD04622-01) | 0.3 | 0.2 |
| 87475 Kidney NAT (OD04622-03) | 2.3 | 2.4 |
| 85973 Kidney Cancer (OD04450-01) | 9.2 | 8.5 |
| 85974 Kidney NAT (OD04450-03) | 1.5 | 1.5 |
| Kidney Cancer Clontech 8120607 | 33.2 | 30.8 |
| Kidney NAT Clontech 8120608 | 1.7 | 2.1 |
| Kidney Cancer Clontech 8120613 | 0 | 0 |
| Kidney NAT Clontech 8120614 | 0.9 | 0.7 |
| Kidney Cancer Clontech 9010320 | 27.4 | 26.8 |
| Kidney NAT Clontech 9010321 | 2.4 | 2.2 |
| Normal Uterus GENPAK 061018 | 0 | 0 |
| Uterus Cancer GENPAK 064011 | 63.3 | 61.6 |
| Normal Thyroid Clontech A+ 6570-1 | 1.7 | 1.6 |
| Thyroid Cancer GENPAK 064010 | 13.8 | 12.3 |
| Thyroid Cancer INVITROGEN A302152 | 1.3 | 1 |
| Thyroid NAT INVITROGEN A302153 | 0.5 | 0.5 |
| Normal Breast GENPAK 061019 | 5.5 | 5.4 |
| 84877 Breast Cancer (OD04566) | 0 | 0 |
| 85975 Breast Cancer (OD04590-01) | 0.9 | 0.9 |
| 85976 Breast Cancer Mets (OD04590-03) | 0.7 | 0.8 |
| 87070 Breast Cancer Metastasis | 0.1 | 0.2 |
| (OD04655-05) | | |
| GENPAK Breast Cancer 064006 | 1.2 | 0.9 |
| Breast Cancer Res. Gen. 1024 | 4.1 | 3.7 |
| Breast Cancer Clontech 9100266 | 1.7 | 1.6 |
| Breast NAT Clontech 9100265 | 1.6 | 1.3 |
| Breast Cancer INVITROGEN A209073 | 12.9 | 12.4 |
| Breast NAT INVITROGEN A2090734 | 6.1 | 6 |
| Normal Liver GENPAK 061009 | 1 | 1 |
| Liver Cancer GENPAK 064003 | 14.4 | 14.2 |
| Liver Cancer Research Genetics RNA | 2.5 | 2.4 |
| 1025 | | |
| Liver Cancer Research Genetics RNA | 4.2 | 4.7 |
| 1026 | | |
| Paired Liver Cancer Tissue Research | 5.3 | 4.7 |
| Genetics RNA 6004-T | | |
| Paired Liver Tissue Research Genetics | 0.1 | 0.1 |
| RNA 6004-N | | |
| Paired Liver Cancer Tissue Research | 5.1 | 4.8 |
| Genetics RNA 6005-T | | |
| Paired Liver Tissue Research Genetics | 1.4 | 1.4 |
| RNA 6005-N | | |
| Normal Bladder GENPAK 061001 | 2.7 | 2.3 |
| Bladder Cancer Research Genetics RNA | 2.7 | 2.8 |
| 1023 | | |
| Bladder Cancer INVITROGEN A302173 | 8.2 | 7.3 |
| 87071 Bladder Cancer (OD04718-01) | 2 | 1.8 |
| 87072 Bladder Normal Adjacent | 0.9 | 0.9 |
| (OD04718-03) | | |
| Normal Ovary Res. Gen. | 0.6 | 0.5 |
| Ovarian Cancer GENPAK 064008 | 100 | 100 |
| 87492 Ovary Cancer (OD04768-07) | 21.9 | 20.7 |
| 87493 Ovary NAT (OD04768-08) | 4.1 | 3.7 |
| Normal Stomach GENPAK 061017 | 2.3 | 2 |
| Gastric Cancer Clontech 9060358 | 0.5 | 0.4 |
| NAT Stomach Clontech 9060359 | 2.6 | 2.2 |
| Gastric Cancer Clontech 9060395 | 5.4 | 5.7 |
| NAT Stomach Clontech 9060394 | 4.9 | 4.7 |
| Gastric Cancer Clontech 9060397 | 14.1 | 13.9 |
| NAT Stomach Clontech 9060396 | 5.1 | 4.4 |
| Gastric Cancer GENPAK 064005 | 0.2 | 0.2 |

TABLE 5
Taq Man Results for Panel 4

| | Rel. Expr., % |
|---|---|
| | 4dtm4832f_{—} |
| Tissue Name | ag588 |
| 93768_Secondary Th1_anti-CD28/anti-CD3 | 0 |
| 93769_Secondary Th2_anti-CD28/anti-CD3 | 0 |
| 93770_Secondary Tr1_anti-CD28/anti-CD3 | 0 |
| 93573_Secondary Th1_resting day 4-6 in IL-2 | 0 |
| 93572_Secondary Th2_resting day 4-6 in IL-2 | 0 |
| 93571_Secondary Tr1_resting day 4-6 in IL-2 | 0 |
| 93568_primary Th1_anti-CD28/anti-CD3 | 0 |
| 93569_primary Th2_anti-CD28/anti-CD3 | 0 |
| 93570_primary Tr1_anti-CD28/anti-CD3 | 0 |
| 93565_primary Th1_resting dy 4-6 in IL-2 | 0 |
| 93566_primary Th2_resting dy 4-6 in IL-2 | 0 |
| 93567_primary Tr1_resting dy 4-6 in IL-2 | 0 |
| 93351_CD45RA CD4 lymphocyte_anti-CD28/anti-CD3 | 0 |
| 93352_CD45RO CD4 lymphocyte_anti-CD28/anti-CD3 | 0 |
| 93251_CD8 Lymphocytes_anti-CD28/anti-CD3 | 1.4 |
| 93353_chronic CD8 Lymphocytes 2ry_resting dy 4-6 in | 0 |
| IL-2 | |
| 93574_chronic CD8 Lymphocytes 2ry_activated | 0 |
| CD3/CD28 | |
| 93354_CD4_none | 0 |
| 93252_Secondary Th1/Th2/Tr1_anti-CD95 CH11 | 0 |
| 93103_LAK cells_resting | 0 |
| 93788_LAK cells_IL-2 | 0 |
| 93787_LAK cells_IL-2 + IL-12 | 0 |
| 93789_LAK cells_IL-2 + IFN gamma | 0 |
| 93790_LAK cells_IL-2 + IL-18 | 0 |
| 93104_LAK cells_PMA/ionomycin and IL-18 | 0 |
| 93578_NK Cells IL-2_resting | 0 |
| 93109_Mixed Lymphocyte Reaction_Two Way MLR | 0 |
| 93110_Mixed Lymphocyte Reaction_Two Way MLR | 0 |
| 93111_Mixed Lymphocyte Reaction_Two Way MLR | 0 |
| 93112_Mononuclear Cells (PBMCs)_resting | 0 |
| 93113_Mononuclear Cells (PBMCs)_PWM | 0.2 |
| 93114_Mononuclear Cells (PBMCs)_PHA-L | 0 |
| 93249_Ramos (B cell)_none | 0 |
| 93250_Ramos (B cell)_ionomycin | 0 |
| 93349_B lymphocytes_PWM | 0.2 |
| 93350_B lymphoytes_CD40L and IL-4 | 0 |
| 92665_EOL-1 (Eosinophil)_dbcAMP differentiated | 0.2 |
| 93248_EOL-1 (Eosinophil)_dbcAMP/PMAionomycin | 0 |
| 93356_Dendritic Cells_none | 0 |
| 93355_Dendritic Cells_LPS 100 ng/ml | 0 |
| 93775_Dendritic Cells_anti-CD40 | 0 |
| 93774_Monocytes_resting | 0 |
| 93776_Monocytes_LPS 50 ng/ml | 0 |
| 93581_Macrophages_resting | 0 |
| 93582_Macrophages_LPS 100 ng/ml | 0 |
| 93098_HUVEC (Endothelial)_none | 0 |
| 93099_HUVEC (Endothelial)_starved | 0 |
| 93100_HUVEC (Endothelial)_IL-1b | 0 |
| 93779_HUVEC (Endothelial)_IFN gamma | 0 |
| 93102_HUVEC (Endothelial)_TNF alpha + IFN gamma | 0 |
| 93101_HUVEC (Endothelial)_TNF alpha + IL4 | 0 |
| 93781_HUVEC (Endothelial)_IL-11 | 0 |
| 93583_Lung Microvascular Endothelial Cells_none | 0 |
| 93584_Lung Microvascular Endothelial Cells_TNFa | 0 |
| (4 ng/ml) and IL1b (1 ng/ml) | |
| 92662_Microvascular Dermal endothelium_none | 0 |
| 92663_Microsvasular Dermal endothelium_TNFa | 0 |
| (4 ng/ml) and IL1b (1 ng/ml) | |
| 93773_Bronchial epithelium_TNFa (4 ng/ml) and | 3.7 |
| IL1b (1 ng/ml)** | |
| 93347_Small Airway Epithelium_none | 53.6 |
| 93348_Small Airway Epithelium_TNFa (4 ng/ml) and | 100 |
| IL1b (1 ng/ml) | |
| 92668_Coronery Artery SMC_resting | 0 |
| 92669_Coronery Artery SMC_TNFa | 0 |
| (4 ng/ml) and IL1b (1 ng/ml) | |
| 93107_astrocytes_resting | 0 |
| 93108_astrocytes_TNFa (4 ng/ml) and IL1b (1 ng/ml) | 0.9 |
| 92666_KU-812 (Basophil)_resting | 0 |
| 92667_KU-812 (Basophil)_PMA/ionoycin | 0 |
| 93579_CCD1106 (Keratinocytes)_none | 0.7 |
| 93580_CCD1106 (Keratinocytes)_TNFa and IFNg** | 0.6 |
| 93791_Liver Cirrhosis | 1.7 |
| 93792_Lupus Kidney | 9.9 |
| 93577_NCI-H292 | 49 |
| 93358_NCI-H292_IL-4 | 61.6 |
| 93360_NCI-H292_IL-9 | 83.5 |
| 93359_NCI-H292_IL-13 | 37.4 |
| 93357_NCI-H292_IFN gamma | 43.2 |
| 93777_HPAEC_- | 0 |
| 93778_HPAEC_IL-1 beta/TNA alpha | 0 |
| 93254_Normal Human Lung Fibroblast_none | 0 |
| 93253_Normal Human Lung Fibroblast_TNFa | 0 |
| (4 ng/ml) and IL-1b (1 ng/ml) | |
| 93257_Normal Human Lung Fibroblast_IL-4 | 0 |
| 93256_Normal Human Lung Fibroblast_IL-9 | 0 |
| 93255_Normal Human Lung Fibroblast_IL-13 | 0 |
| 93258_Normal Human Lung Fibroblast_IFN gamma | 0 |
| 93106_Dermal Fibroblasts CCD1070_resting | 0 |
| 93361_Dermal Fibroblasts CCD1070_TNF alpha | 0 |
| 4 ng/ml | |
| 93105_Dermal Fibroblasts CCD1070_IL-1 beta 1 | 0 |
| ng/ml | |
| 93772_dermal fibroblast_IFN gamma | 0 |
| 93771_dermal fibroblast_IL-4 | 0 |
| 93259_IBD Colitis 1** | 0.1 |
| 93260_IBD Colitis 2 | 0 |
| 93261_IBD Crohns | 0 |
| 735010_Colon_normal | 0.7 |
| 735019_Lung_none | 36.3 |
| 64028-1_Thymus_none | 1.4 |
| 64030-1_Kidney_none | 3.9 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a cancer cell comprising:
a) measuring expression of a nucleic acid encoding an antileukoprotease polypeptide in a test sample; and
b) comparing the expression of the nucleic acid in the test sample to the expression of a nucleic acid encoding an antileukoprotease polypeptide in a cancer reference profile, wherein a similarity between the expression of the nucleic acid in the test sample and the reference profile indicates the presence of a cancer cell in the test sample.

2. The method of claim 1, wherein the cancer is selected from the group consisting of ovarian cancer, thyroid cancer, and renal cancer

3. The method of claim 2, wherein the cancer is ovarian.

4. A method of identifying a cancer cell comprising:
a) measuring expression of a nucleic acid encoding an antileukoprotease polypeptide in a test sample; and
b) comparing the expression of the nucleic acid in the test sample to the expression of a nucleic acid encoding an antileukoprotease polypeptide in a normal reference profile,
wherein an increase in expression of the nucleic acid in the test sample compared to the normal reference profile indicates the presence of a cancer cell in the test sample.

5. The method of claim 4, wherein the cancer is selected from the group consisting of ovarian cancer, thyroid cancer, and renal cancer

6. The method of claim 5, wherein the cancer is ovarian.

7. The method of claim 1, wherein the nucleic acid comprises the sequence of SEQ ID NO:1.

8. The method of claim 1, wherein the nucleic acid encoding an antileukoprotease polypeptide comprises the amino acid sequence of SEQ ID NO:2.


SEQUENCE LISTING (5 sequences)

SEQ ID NO: 1 - DNA, 594 bp, Homo sapiens
gtcactcctgccttcaccatgaagtccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttgatcaatgaataaataaacgagcctatttctctttgcac

SEQ ID NO: 2 - protein, 132 aa, Homo sapiens
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 3 - DNA, 20 bp, Artificial Sequence
  Description of Artificial Sequence Oligonucleotide primer
tgccttcaccatgaagtcca

SEQ ID NO: 4 - DNA, 19 bp, Artificial Sequence
  Description of Artificial Sequence Oligonucleotide primer
agcccaaggtgccagagtt

SEQ ID NO: 5 - DNA, 23 bp, Artificial Sequence
  Description of Artificial Sequence Oligonucleotide primer
cttcctggtgctgcttgccctgg